(19) United States
(12) Patent Application Publication
(10) Pub. No.: US 2022/0142520 A1
(43) Pub. Date: May 12, 2022

(54) A NON-INVASIVE GLUCOMETER

(71) Applicant: Biofi Medical Healthcare India Private Limited, Bangalore (IN)

(72) Inventors: Surya Prakash Maguluri, Medak (IN); Ravikumar Rajendraprasad, Bangalore (IN); Amutha Devi Ravikumar, Bangalore (IN); Pauldurai Shanmugam, Tirunelveli (IN); Girimaji Sathyanarayana Rao Ravi, Bangalore (IN)

(73) Assignee: Biofi Medical Healthcare India Pvt Ltd., Bangalore (IN)

(21) Appl. No.: 17/438,882

(22) PCT Filed: Mar. 13, 2020
(86) PCT No.: PCT/IN2020/050229

§ 371 Date: Sep. 13, 2021

(30) Foreign Application Priority Data
Mar. 13, 2019 (IN) 201941009757

(51) Int. Cl. A61B 5/145; A61B 5/00; A61B 5/021; A61B 5/1455

(52) U.S. Cl. CPC A61B 5/14532; A61B 5/6826; A61B 5/7264; A61B 2562/046; A61B 5/02116; A61B 5/1455; A61B 5/0059; A61B 5/742

(57) ABSTRACT

A glucometer for monitoring blood glucose comprises a curved test surface with a transparent test site. The curved test surface accommodates a finger-tip and an enclosing lid encloses the finger-tip when it is placed on the transparent test site. The glucometer further comprises a light source assembly comprising light emitting diodes, underneath the transparent test site. The glucometer further comprises detectors for detecting transflective light, transmissive light, and reflective light from blood capillaries at a depth of 100 micro meters below skin surface. The glucometer further comprises a software module comprising a first sub-module that comprises a first set of algorithms for generating a data matrix. The transflective, transmissive light, and reflective light from blood capillaries are measured and recorded in data matrix. A second sub-module comprises a second set of algorithms for non-invasive measurement of concentration of glucose in the blood by processing data within the data matrix.

### CROSS-REFERENCE TO RELATED APPLICATIONS

This application is a PCT application that claims priority to and the benefit of the provisional patent application titled “A Non-Invasive Diabetic Profiler”, application number 201941009757, filed in the Indian Patent Office on Mar. 13, 2019. The specification of the above referenced patent application is incorporated herein by reference in its entirety.

### TECHNICAL FIELD OF THE INVENTION

The present invention relates to a non-invasive glucometer for monitoring glucose levels in blood. More specifically, the present invention relates to a non-invasive glucometer and method for performing data analytics and trends over the period of time, on the data obtained from the glucometer, using artificial intelligence-based analytics. More specifically, the invention relates to a non-invasive glucometer for monitoring blood glucose levels using visible and Near-Infrared (NIR) light source and the risk of onset of other metabolic conditions associated with diabetes and for prediction of risk of onset of diabetes in non-diabetics.

### BACKGROUND

The blood sugar test measures the sugar or glucose levels in the blood. Specially, in diabetic patients, it is important to monitor the glucose level and to maintain the levels within the controlled range. The test result determines the glucose levels in the blood, the effectiveness of the medication and treatments in diabetic patients.

The glucometer is a handy device to test blood glucose level for patients requiring regular monitoring of blood glucose as a test kit device. The device widely used is an invasive blood glucose level monitor. A drop of blood is placed on the test strip by means of a lancet to prick the fingertip of the diabetic patient. The test strip is placed in the meter reader, which displays the results within few seconds.

Alternatively, the availability of the advanced devices allows the diabetic patients to test the blood glucose levels from region such as upper arm, forearm, the palm, base of the thumb and thigh. However, the results vary in contrast to the results provided by the fingertip region or capillary blood. Moreover, the results are also not as accurate as measured from fingertip samples when there is fluctuation in the blood sugar level especially in patients with long-term diabetes.

Additionally, the use of sensors by placing them under the skin to measure the blood sugar level for continuous glucose testing, which transmits the reading to a recording device worn by the diabetic patients sounds an alarm when the blood glucose levels increase. However, the sensors are expensive and needs to be replaced frequently.

Diagnosis of diabetes and monitoring of blood glucose level is uneconomical due to recurring costs of the test strips and lancets. The test kits display low level compliance since patient is required to prick the finger with the lancet and place a drop of blood on the test strip each time for measuring the glucose levels. Sometimes, the pricking also leads to vascular infections. Moreover, diabetic complications and their diagnosis adds as an additional cost, to the already uneconomical situations. Currently, the invasive and minimally invasive blood glucose monitors are available and majority of them are standalone devices which does not include corrections for temperature, humidity, altitude variations which affects the glucose readings.

Diabetes is associated with many other metabolic diseases. The long-term persistence of diabetes results in metabolic complications related to organs such as eyes, kidneys, liver etc. It is required to monitor the onset of other metabolic complications in addition to continuous monitoring of blood glucose level in diabetic patients.

Diabetes is a hereditary condition and the onset of diabetes is expected in non-diabetic population with a history of diabetic hereditary from ancestors. There is no device available to analyze the risk of onset of diabetes.

The exiting methods to detect the blood glucose levels are not economical, the invasive blood glucose detectors have huge recurring cost and create discomfort to the user. The regular pricking for collecting the blood sample further leads to vascular infections. Additionally, the non-invasive devices require regular calibrations. Most of the available devices for blood glucose monitoring are suitable for a single user since the device cannot be used for multiple users because of hygienic reasons, data collusion and calibration issues. The device provides the reading of the blood glucose levels but does not address to the possible way to manage the diabetic condition. The devices also do not provide the details of onset of diabetes in non-diabetics.

Hence, in order to overcome the disadvantages that exist in the state of the art devices available in the market, there is need of a non-invasive device that can read glucose level along with detecting the predisposed condition with improper management of diabetes.

### SUMMARY OF THE INVENTION

This summary is provided to introduce a selection of concepts in a simplified form that are further disclosed in the detailed description of the invention. This summary is not intended to determine the scope of the claimed subject matter.

A glucometer for monitoring blood glucose is provided. The glucometer comprises a curved test surface with a transparent test site. The curved test surface is configured to accommodate a finger-tip when the finger-tip is placed on the transparent test site for non-invasively measuring concentration of glucose in blood. The glucometer further comprises an enclosing lid configured to enclose the finger-tip placed on the transparent test site. The glucometer further comprises a light source assembly comprising light emitting diodes (LEDs), placed underneath the transparent test site. The glucometer further comprises multiple detectors for detecting transflective light, transmissive light, and reflective light from blood capillaries at a depth of 100 micro meters below skin surface. The glucometer further comprises a software module comprising two sub-modules. A first of the two sub-modules comprises a first set of algorithms for generating a data matrix. Each of the plurality of light emitting diodes is configured to be individually activated in a loop comprising 1200 cycles. The transflective light, the transmissive light, and the reflective light from the blood capillaries are measured and recorded in the data matrix of dimension (n×1200×m), where ‘n’ is a number of distinct wavelengths of light emitting diodes within the light source assembly and ‘m’ is a number of detectors. A second of the two sub-modules comprises a second set of algorithms for the non-invasive measurement of the concentration of the glucose in the blood by processing data within the data matrix.

A method of measuring concentration of glucose in blood comprises providing a glucometer comprising a curved test surface with a transparent test site, an enclosing lid, a light source assembly, and a plurality of detectors for detecting transflective light, transmissive light, and reflective light from blood capillaries at a depth of 100 micro meters below skin surface, and a software module. The light source assembly comprises multiple light emitting diodes placed underneath the transparent test site. The software module comprises a first sub-module and a second sub-module. The first sub-module of the software module comprises a first set of algorithms for generating a data matrix and the second sub-module comprises a second set of algorithms for the non-invasive measurement of the concentration of the glucose in the blood by processing data within the data matrix. The method further comprises applying pressure to a finger-tip placed on the transparent test site by closing the enclosing lid on the finger-tip; activating each of the light emitting diodes individually in a loop comprising 1200 cycles; measuring the transflective light, the transmissive light and the reflective light from the blood capillaries within the pressurized finger-tip, and recording the measurements in the data matrix of dimension (n×1200×m), where ‘n’ is a number of distinct wavelengths of the light emitting diodes within the light source assembly and ‘m’ is a number of detectors; and measuring the concentration of the glucose in the blood by the processing of the data within the data matrix.

In one or more embodiments, related systems comprise circuitry and/or programming for effecting the methods disclosed herein. The circuitry and/or programming can be any combination of hardware, software, and/or firmware configured to effect the methods disclosed herein depending upon the design choices of a system designer. Also, in an embodiment, various structural elements can be employed depending on the design choices of the system designer.

### BRIEF DESCRIPTION OF DRAWINGS

The foregoing summary, as well as the following detailed description of the invention, is better understood when read in conjunction with the appended drawings. For illustrating the invention, exemplary constructions of the invention are shown in the drawings. However, the invention is not limited to the specific methods and components disclosed herein. The description of a method step or a component referenced by a numeral in a drawing is applicable to the description of that method step or component shown by that same numeral in any subsequent drawing herein.

FIG. 1 illustrates a glucometer for non-invasively measuring concentration of glucose in blood.

FIG. 2 illustrates a diagram showing LEDs placed underneath a transparent test site and detectors arranged along a curved test surface on both sides of the transparent test site.

FIG. 3 illustrates a system architecture diagram of the glucometer.

FIGS. 4A-4B illustrate a method of non-invasively measuring concentration of glucose in blood.

FIG. 5A illustrates a flowchart showing a reading cycle of the detectors for each of the LEDs.

FIG. 5B illustrates a data matrix of dimension (n×1200×m) where transflective light, transmissive light, and reflective light from blood capillaries are recorded.

FIG. 6 illustrates an algorithm for processing a data matrix.

FIG. 7 illustrates a flow chart for applying the statistical processing of the absorbance data and displaying the concentration of blood glucose value as output.

FIG. 8 illustrates the artificial neural network and error correction module.

FIG. 9 illustrates an algorithm used to train the artificial neural network and error correction module.

### DETAILED DESCRIPTION OF THE INVENTION

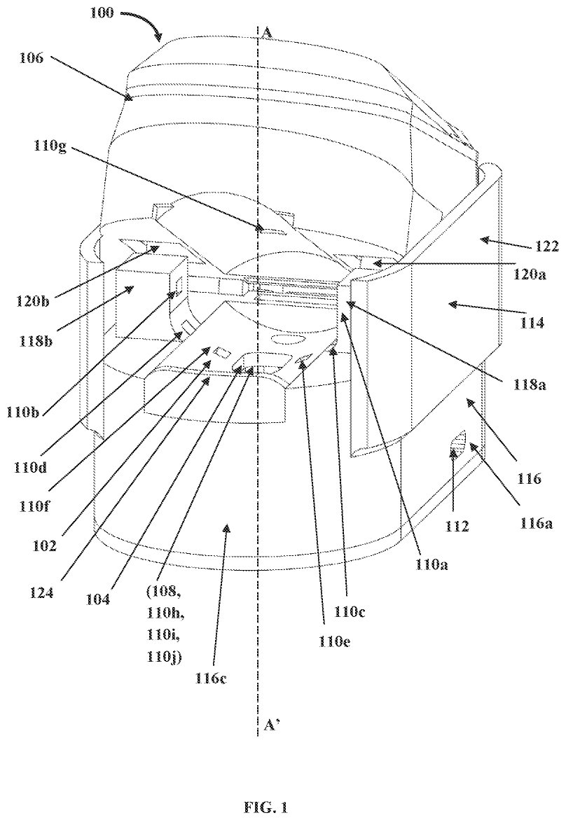

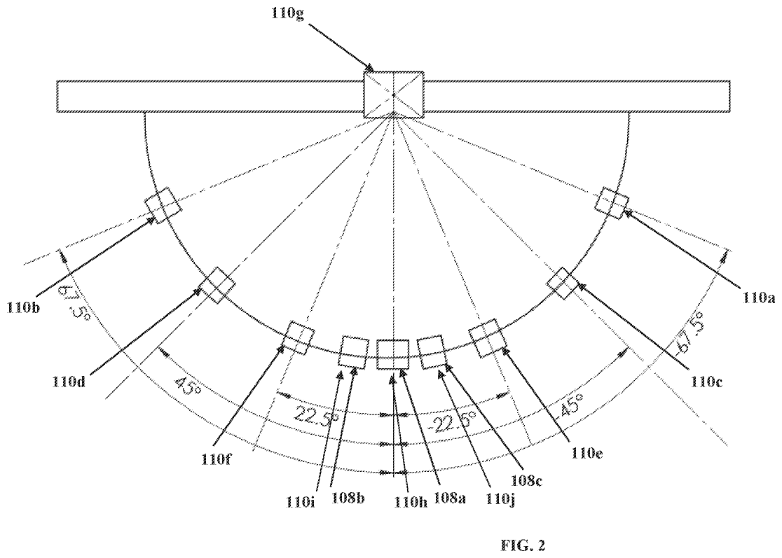

FIG. 1 illustrates an embodiment of a glucometer 100 for non-invasively measuring concentration of glucose in blood. The glucometer 100 comprises a curved test surface 102 with a transparent test site 104. The curved test surface 102 is configured to accommodate a finger-tip when the finger-tip is placed on the transparent test site 104 for non-invasively measuring concentration of glucose in blood. As used herein, the term “Non-invasive” refers to procedure that does not require insertion of an instrument or device through the skin or a body orifice for diagnosis or treatment. The glucometer 100 further comprises an enclosing lid 106 configured to enclose the finger-tip placed on the transparent test site 104. The glucometer 100 further comprises a light source assembly 108 comprising multiple light emitting diodes (LEDs) 108a-108c, as illustrated in FIG. 2, placed underneath the transparent test site 104. The glucometer 100 further comprises detectors 110a-110j for detecting transflective light, transmissive light, and reflective light from blood capillaries at a depth of 100 micro meters below skin surface. The glucometer 100 further comprises a software module 306 comprising two sub-modules, namely a first sub-module 308 and a second sub-module 310. The first sub-module 308 comprises a first set of algorithms for generating a data matrix.

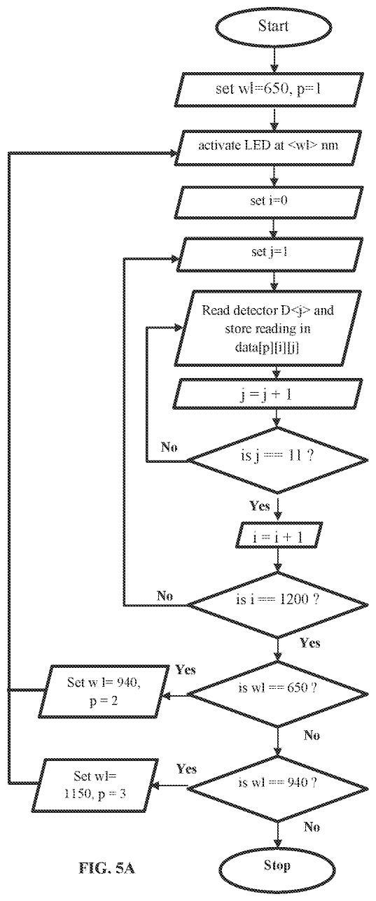

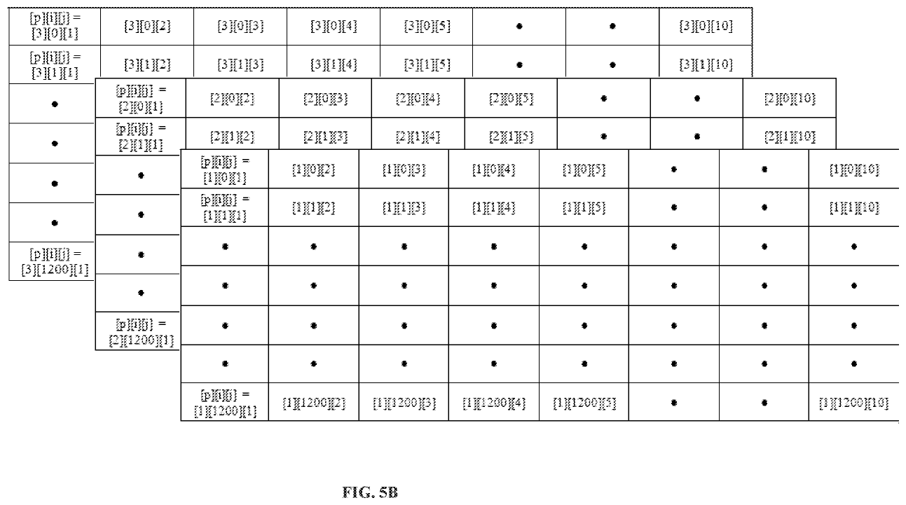

Each of the plurality of light emitting diodes 108a-108c is configured to be individually activated in a loop comprising 1200 cycles and the transflective light, the transmissive light, and the reflective light from the blood capillaries are measured and recorded in the data matrix of dimension (n×1200×m). In the matrix dimension, ‘n’ is a number of distinct wavelengths of light emitting diodes 108a-108c within the light source assembly 108 and ‘m’ is a number of detectors 110a-110j. The initial value of all entries in the data matrix, exemplarily illustrated in FIG. 5B are set to zero. In an embodiment, the glucometer 100 is configured to set the initial value of all entries in the data matrix to zero prior to each reading. The second sub-module 310 in the software module 306 comprises a second set of algorithms for the non-invasively measuring the concentration of glucose in the blood by processing data within the data matrix.

In an embodiment, the curved test surface 102 is hemispherical in shape, as illustrated in FIG. 1A. The curved test surface 102 extends from the location of the first detector 110a to the location of the sixth detector 110b, as illustrated in FIG. 1A. In another embodiment, curved test surface 102 has one of a parabolic shape, a hyperbolic shape, an arc shape, etc. In an embodiment, the curved test surface 102 is integrally formed on a body 116 of the glucometer 100, as illustrated in FIG. 1A. The light emitting diodes 108a-108c, the detectors 110a-110f, and detectors 110h-110j are housed within the body 116 of the glucometer 100, whereas the detector 110g is housed in the enclosing lid 106. In an embodiment, the enclosing lid 106 is configured to apply pressure to the finger-tip placed on the transparent test site 104. The enclosing lid 106 is attached to the body 116 through a hinge 114. In an embodiment, the hinge 114 is spring loaded. The hinge 114 is configured to apply a predetermined amount of pressure on the finger placed on the transparent test site 104. In an embodiment, portion 118a of the curved test surface 102 that houses detectors 110a and 110c, and portion 118b of the curved test surface 102 that houses detectors 110d and 110d, protrude above from the body 116 of the glucometer 100. The enclosing lid 106 comprises cavities 120a and 120b adapted to receive and enclose the protruding portions 118a and 118b of the curved test surface 102. The glucometer 100 further comprises a power input, for example a USB port 112 for receiving input power from a power adapter. The USB port 112 is one of a Type A USB port, a Type B USB port, and a Type C USB port.

In an embodiment, the glucometer 100 further comprises a guard member 122, as illustrated in FIG. 1. The guard member 122 surrounds a right side 116a and a left side 116b of the body 116 of the glucometer 100 adjacent to the curved test surface 102 of the glucometer 100 to block light from reaching the curved test surface 102 from the right side 116a and the left side 116b of the glucometer 100. In an embodiment, the guard member 122 is configured to slidably engage with the body 116 of the glucometer 100. In an embodiment, the guard member 122 surrounds the right side 116a, a rear side 116d, and the left side 116b of the glucometer 100. In an embodiment, the guard member 122 does not surround a front side 116c of the glucometer 100. In an embodiment, the glucometer 100 further comprises a finger occlusion device 124 located at the front side 116c of the glucometer 100, adjacent to the curved test surface 102. The finger occlusion device 124 projects above the curved test surface 102 and is configured to block the blood flow to and from the finger-tip, to enhance the optical properties of the finger-tip, when the enclosing lid 106 applies pressure on the finger-tip placed on the transparent test site 104. When a user places his finger-tip on the transparent test site 104 of the curved test surface 102, the user's distal interphalangeal joint generally lies on the finger occlusion device 124. The projection of the finger occlusion device 124 is configured to block the blood flow through the distal interphalangeal joint of the user's finger-tip.

FIG. 2 illustrates a diagram showing LEDs places underneath a transparent test site and detectors arranged along a curved test surface on both sides of the transparent test site. As mentioned above, the light source assembly 108 comprises multiple LEDs 108a-108c. In an embodiment, the light source assembly 108 comprises three distinct wavelengths of light emitting diodes (LEDs) 108a-108c. Therefore, the value of ‘n’ in the dimension of the data matrix ranges from 1 to 3. A first of the distinct LEDs, for example, 108a, is a red LED operating with a wavelength of 650 nanometers, a second of the distinct LEDs, for example, 108b, is a near-infrared (NIR) LED operating at a wavelength of 940 nanometers, and a third of the distinct light emitting diodes, for example, 108c, is a near-infrared (NIR) LED operating at a wavelength of 1160 nanometers. In an embodiment, alternative light sources, for example, lasers, light bulbs, etc., capable of emitting visible light and near-infrared light are used in place of LEDs. In an embodiment, the glucometer 100 comprises ten detectors 110a-110j. Therefore, the value of ‘m’ in the dimension of the data matrix ranges from 1 to 10. The ten detectors comprise a first detector set comprising six detectors 110a-110f for detecting the transflective light, a second detector set comprising a detector 110g for detecting the transmissive light, and a third detector set comprising three detectors 110h-110j for detecting the reflective light. The detectors 110a-110f in the first detector set are arranged along the curved test surface 102 on both sides of the transparent test site 104. A seventh detector 110g in the second detector set is accommodated in the enclosing lid 106 along a longitudinal axis A-A′ perpendicular to a center of the transparent test site 104, and wherein the detectors 110h-110j in the third detector set comprising an eighth detector 110h, a ninth detector 110i, and a tenth detector 110j are accommodated within the light source assembly 108.

A first detector 110a and a sixth detector 110f of the first detector set are placed at an angle of −67.5° and +67.5° from the longitudinal axis linking the center of the transparent test site 104 and the seventh detector 110g. A a second detector 110b and a fifth detector 110e of the first detector set are placed at an angle of −45° and +45° from the longitudinal axis linking the center of the transparent test site 104 and the seventh detector 110g. Also a third detector 110c and a fourth detector 110d of the first detector set are placed at an angle of −22.5° and +22.5° from the longitudinal axis linking the center of the transparent test site 104 and the seventh detector 110g. The eighth detector 110h is co-located with the first light emitting diode 108a, the ninth detector 110i is co-located with the second light emitting diode 108b, and the tenth detector 110j is co-located with the third light emitting diode 108c. In an embodiment, the eighth detector 110h, the ninth detector 110i, tenth detector 110j, the first light emitting diode 108a, the second light emitting diode 108b, and the third light emitting diode 108c are co-located with each other. In an embodiment, the distance between a detector or a light emitting diode and a co-located light emitting or a detector is about 1 mm to about 4 mm.

The seventh detector 110g detects the transmissive light passing through the finger-tip and through blood capillaries at the depth of 100 micro meters below the skin surface. wherein the first detector 110a, the second detector 110b, the third detector 110c, the fourth detector 110d, the fifth detector 110e and the sixth detector 110f detect the transflective light reflected from the blood capillaries, and wherein the eighth detector 110h, the ninth detector 110i and the tenth detector 110j detect the reflective light from the blood capillaries.

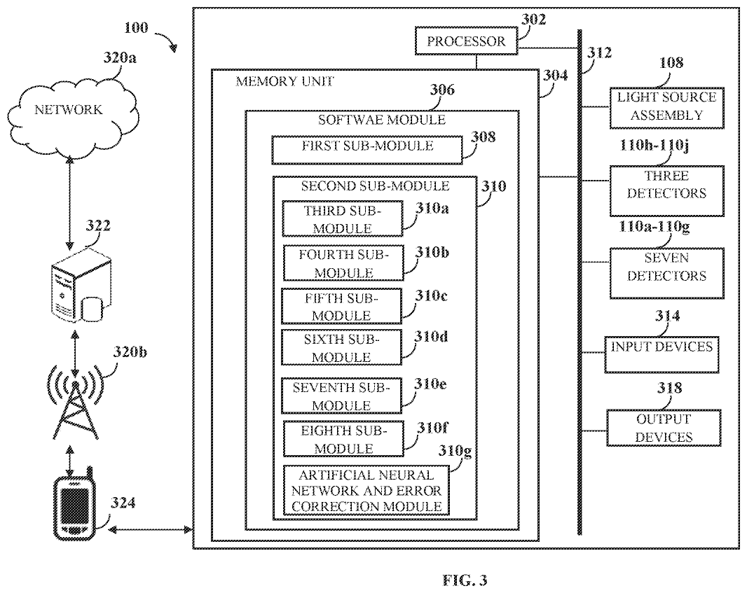

FIG. 3 illustrates a system architecture diagram of the glucometer 100. As illustrated in FIG. 3, the glucometer 100 comprises a processor 302, a memory unit 304, a two-way communication interface 312, for example, a communication bus 312, input devices 314, output devices 318, the light source assembly 108 comprising the LEDs 108a-108c, and the detectors 110a-110j. The input devices 314, output devices 318, the light source assembly 108 comprising the LEDs 108a-108c, and the detectors 110a-110j are connected to the memory unit 304 and the processor 302 through the communication interface 312.

In an embodiment, the software module 306 is located inside the glucometer 100 which consist of eight sub modules 310a-310f and an Artificial Neural Network and Error correction module 310g. The algorithm in the first sub-module 308 is illustrated in FIG. 5A. The second sub-module 310 of the software module 306 comprises a third sub-module 310a, a fourth sub-module 310b, a fifth sub-module 310c, a sixth sub-module 310d, a seventh sub-module 310e, an eighth sub-module 310f, and an artificial neural network and error correction module 310g.

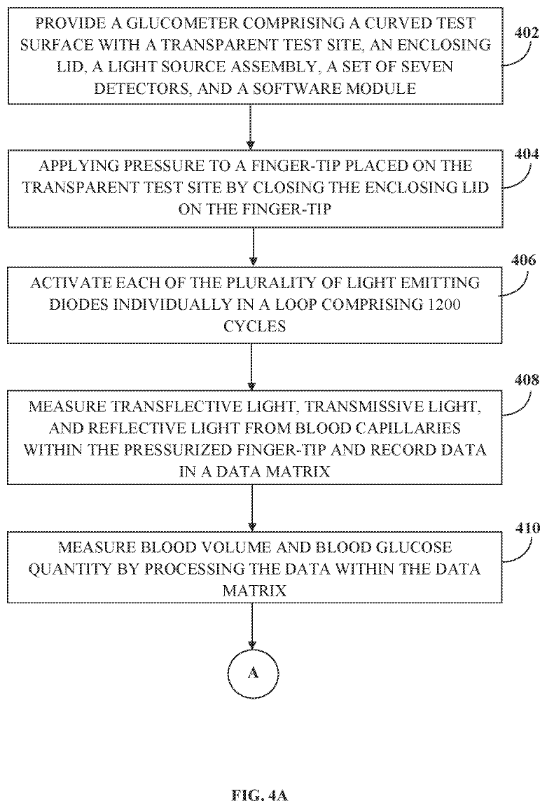

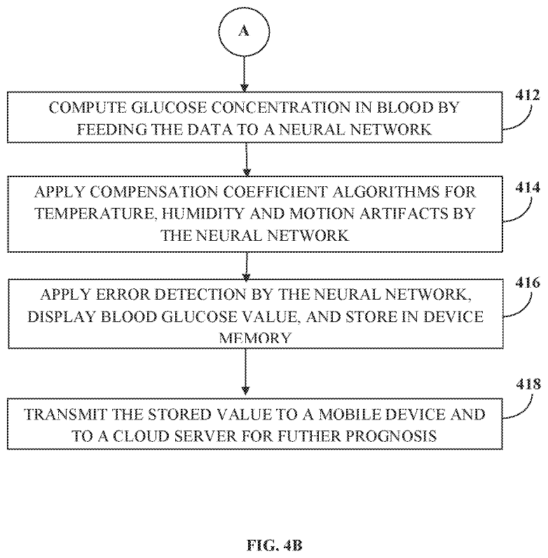

FIGS. 4A-4B illustrate a method of non-invasively measuring concentration of glucose in blood. The method comprises providing 402 the glucometer 100 comprising the curved test surface 102 with the transparent test site 104, the enclosing lid 106, the light source assembly 108, detectors 110a-110j for detecting transflective light, transmissive light, and reflective light from blood capillaries at a depth of 100 micro meters below skin surface, and the software module 306 as illustrated in FIG. 3. The first set of algorithms in the first sub-module 308 generating a data matrix of dimension (n×1200×m), where ‘n’ is the number of distinct wavelengths of the light emitting diodes within the light source assembly and ‘m’ is the number of detectors. Prior to obtaining a reading, the initial values of all entries in the data matrix are set to zero by the first set of algorithms in the first sub-module 308. The method further comprises applying 404 pressure to a finger-tip placed on the transparent test site 102 by closing the enclosing lid 106 on the finger-tip. The method further comprises activating 406 each of the plurality of light emitting diodes individually in a loop comprising 1200 cycles. The method further comprises measuring 408 the transflective light, the transmissive light and the reflective light from the blood capillaries within the pressurized finger-tip, and recording the measurements in the data matrix. The method further comprises measuring 410 the concentration of the glucose in the blood by the processing of the data within the data matrix.

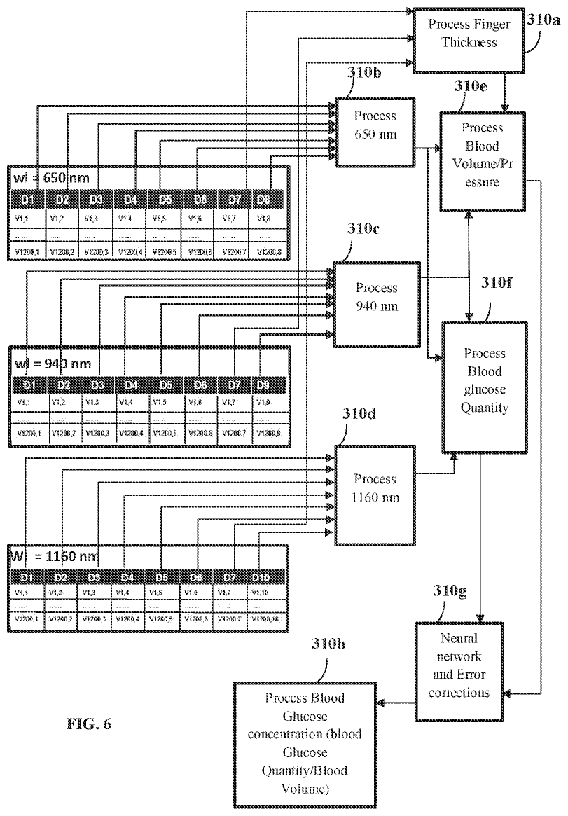

FIG. 5A illustrates a flowchart showing a reading cycle of the detectors for each of the LEDs. Each of the light emitting diodes 108a-108c is configured to be individually activated in a loop comprising 1200 cycles and the transflective light, the transmissive light, and the reflective light from the blood capillaries are measured and recorded in the data matrix of dimension (n×1200×m). The second set of algorithms in the second sub-module 310 measures the concentration of the glucose in the blood by processing data within the data matrix. The algorithm in the second sub-module 310 is illustrated in FIG. 6. Since there are three (3) light emitting diodes (108a, 108b, and 108c) within the light source assembly 108, ‘n’=3 in the data matrix of dimension (n×1200×m). In the three (3) light emitting diodes (108a, 108b, and 108c a first light emitting diode 108a is a red light emitting diode operating with a wavelength of 650 nanometers. A second light emitting diode 108b is a Near-Infrared (NIR) light emitting diode operating at a wavelength of 940 nanometers. A third light emitting diode 108c is a Near-Infrared (NIR) light emitting diode operating at a wavelength of 1160 nanometers.

FIG. 5B illustrates a data matrix where the transflective light, the transmissive light, and the reflective light from the blood capillaries are recorded. The data matrix is, for example, a three dimensional matrix with a dimension of [p][i][j], where p=1, 2, or 3, and corresponds to the number of distinct wavelengths of the light emitting diodes. For example, as illustrated in FIG. 5A, p=1 when the first light emitting diode 108a with a wavelength of 650 nanometers is activated, p=2 when the second light emitting diode 108b with a wavelength of 940 nanometers is activated, and p=3 when the third light emitting diode 108a with a wavelength of 1160 nanometers is activated. In the above data matrix dimension, i=0, 1, 2, . . . , 1200; and j=1, 2, . . . , 10. The value of ‘j’ corresponds to the number of detectors 110a-110j. The detectors 110a-110j stop detecting the light for a selected light emitting diode (108a, 108b, or 108c) when the value of j=11.

As illustrated in FIG. 5A, the first light emitting diode 108a with a wavelength of 650 nanometers is activated first. To obtain the output of the first detector 110a for the first light emitting diode 108a with the wavelength of 650 nanometers, the value of ‘p’ is set to ‘1’, value of T is set to ‘0’, and value of ‘j’ is set to ‘1’. The reading of the first detector 110a is entered in the data matrix at [1][0][1], as shown in FIG. 5B. The value of ‘j’ is incremented by ‘1’ and the reading of the second detector 110b is entered in the data matrix at [1][0][2]. The value of ‘j’ is incremented by ‘1’ till ‘10’ and for each increment, the respective detector is read and data is entered in the data matrix. Next, the second row of the matrix is updated after incrementing the value of ‘i’ by ‘1’. The data is entered in first page of the data matrix until the value of ‘i’=1200 and ‘j’=11. The reading cycle collects 1200 readings from each of the ten detectors 110a-110j for the first light emitting diode 108a. Similarly, in the next reading cycle, ‘p’ is set to 2 and 1200 readings are collected from each of the ten detectors 110a-110j for the second light emitting diode 108b of 940 nanometers wavelength. Likewise, in the next reading cycle, ‘p’ is set to 3 and 1200 readings are collected from each of the ten detectors 110a-110j for the third light emitting diode 108c of 1160 nm wavelength. The sampling rate is fixed at 600 samples/s for each of the light emitting diodes 108a, 108b, and 108c. After the reading cycle is complete, the data matrix with dimension 3×1200×10 is generated, as illustrated in FIG. 5B. The data matrix holds the recorded detector outputs. Where matrix has 3 pages, each page represents data collected for each distinct wavelength of the light emitting diodes. Each page has 10 columns and each column in the page holds 1200 readings from a detector.

The transflective method measurements are available in the data matrix for detectors 110a-110f. The light from the LED illuminates the finger surface and the light penetrates 100 um below the skin. After that, the light is reflected at different angles. The reflected light is captured by detectors 110a-110f. Since the blood capillaries are present at 100 um depth below the skin surface, the light interacts with the blood flow and the intensity of the reflected light is pulsating in nature. The pulsating nature is due to blood volume changes in the blood capillaries due to the blood pumping action of the heart. These pulses have frequency equal to the heart rate of the person and the peak-to-peak amplitude of the pulse is very low as the minor changes in the blood volume causes minute changes in the intensity of light. The detectors at different angles will have different amplitude of the pulses. The detector having the highest peak-to-peak amplitude is most relevant for getting an estimation of blood glucose concentration and the detector having lowest pulse amplitudes are least relevant as these are due to the scattering effect of light.

In an embodiment, the second sub-module 310 comprises an artificial neural network and error correction module 310g for combining the blood volume data and the blood glucose quantity to compute the concentration of the glucose in the blood. The artificial neural network and error correction module 310g further comprise machine learning and has the capability to learn the direction of scattered light and eventually minimizes error caused due to scattering effect.

The transmissive method measurements are available in the ‘data’ matrix for the seventh detector 110g. The light from LEDs passes through the finger and is detected by the seventh detector 110g. The average value of the seventh detector 110g output is proportional to the finger thickness. In an embodiment, the artificial neural network and error correction module 310g involve error cancellation due to finger thickness as the algorithms take finger thickness as a factor. With the help of the seventh detector 110g output, the artificial neural network and error correction module 310g learns the error caused due to finger thickness of a person and compensates for that error.

The reflective method measurements are available in the data matrix for detectors 110h-110j. The light from the LED illuminates the finger surface and the light penetrates 100 um below the skin. After that, the light is reflected directly back. The reflected light is captured by detectors 110h-110j

FIG. 6 illustrates an algorithm for processing the data matrix. The second-sub module 310 of the software module 306 comprises various sub-modules for processing the data. The third sub-module 310a combines data within the data matrix measured by the seventh detector 110g at the wavelengths of 650 nanometers, 940 nanometers and 1160 nanometers to determine skin thickness correction data caused by variation in skin thickness. The fourth sub-module 310b combines data within the data matrix measured by the first detector 110a, the second detector 110b, the third detector 110c, the fourth detector 110d, the fifth detector 110e, the sixth detector 110f and the eighth detector 110h at the wavelength of 650 nanometers. The fifth sub-module 310c combines data within the data matrix measured by the first detector 110a, the second detector 110b, the third detector 110c, the fourth detector 110d, the fifth detector 110e, the sixth detector 110f and the ninth detector 110i at the wavelength of 940 nanometers. The sixth sub-module 310d combines data within the data matrix measured by the first detector 110a, the second detector 110b, the third detector 110c, the fourth detector 110d, the fifth detector 110e, the sixth detector 110f and the tenth detector 110j at the wavelength of 1160 nanometers. The seventh sub-module 310e combines outputs of the third sub-module 310a, the fourth sub-module 310b, and the fifth sub-module 310c for processing one of blood volume and blood pressure data. It is known that a relationship exists between blood volume and blood pressure in humans. It has been recognized that in essential hypertension, renovascular hypertension, and pheochromocytoma, the relationship between plasma volume and diastolic blood pressure is an inverse one. The eighth sub-module 310f combines outputs of the fourth sub-module 310b, the fifth sub-module 310c, and the sixth sub-module 310d to process blood glucose quantity.

As illustrated in FIG. 4B, the method further comprises combining 412 the blood volume data and the blood glucose quantity to compute the concentration of the glucose in the blood, by the artificial neural network and error correction module 310g. The method further comprises applying 414 compensation coefficient algorithms to mitigate effects of temperature, humidity, and motion artifacts, by the artificial neural network and error correction module 310g. The method further comprises applying 416 error detection, by the artificial neural network and error correction module 310g, before the computed concentration of the glucose in the blood is displayed on the display screen 310h of the glucometer 100. The method further comprises storing 416 the computed concentration of the glucose in the blood in a memory of the glucometer 100 and transmitting 418 the stored data to a mobile device and to a cloud server for further prognosis.

In an embodiment, one or more of the determined skin thickness correction data, blood glucose data, blood volume data, and the concentration of the glucose in the blood are displayed on an output device 318, for example a display unit. The glucometer 100 further comprises one or more input devices 314, for example, one or more input buttons to allow a user to enter information comprising a user name, age, etc.

In a second embodiment, the glucometer 100 comprises four (4) detectors (110g-110j) for detecting transmissive light and reflective light from blood capillaries at a depth of 100 micro meters below skin surface. The glucometer 100 further comprises a curved test surface 102 with a transparent test site 104. The curved test surface 102 is configured to accommodate a finger-tip when the finger-tip is placed on the transparent test site 104 for non-invasively measuring concentration of glucose in blood. The glucometer 100 further comprises an enclosing lid 106 configured to enclose the finger-tip placed on the transparent test site 104. The glucometer 100 further comprises a light source assembly 108 comprising multiple light emitting diodes (LEDs) 108a-108c, placed underneath the transparent test site 104. The four detectors (110g-110j) comprise a first detector 110g for detecting transmissive light; and a second detector 110h, a third detector 110i, and a fourth detector 110j for detecting the reflective light. The first detector 110g is accommodated in the enclosing lid 106 along a longitudinal axis perpendicular to a center of the transparent test site 104, and the detectors (110h-110j) are accommodated within the light source assembly 108, as illustrated in FIG. 1. The glucometer 100 further comprises a software module 306 comprising two sub-modules, namely a first sub-module 308 and a second sub-module 310. The first sub-module 308 comprises a first set of algorithms for generating a data matrix.

Each of the plurality of light emitting diodes (108a-108c) is individually activated in a loop comprising 1200 cycles and the transmissive light and the reflective light from the blood capillaries are measured and recorded in the data matrix of dimension (n×1200×m). In this second embodiment of the glucometer 100, ‘n’=3, and ‘m’=4. The second detector 110h is co-located with the first light emitting diode 108a, the third detector 110i is co-located with the second light emitting diode 108b, the fourth detector 110j is co-located with the third light emitting diode 108c. The first detector 110g detects the transmissive light passing through the finger-tip and through blood capillaries at the depth of 100 micro meters below the skin surface. The second detector 110h, the third detector 110i, and the fourth detector 110j detect the reflective light reflected from the blood capillaries.

The second sub-module 310 of the software module 306 comprises a third sub-module 310a for using the data within the data matrix measured by the first detector 110g at the wavelengths of 650 nanometers to determine skin thickness correction data caused by variation in skin thickness. The second sub-module 310 of the software module 306 comprises a fourth sub-module 310b for combining data within the data matrix measured by the second detector 110h, the third detector 110i, and the fourth detector 110i to process blood glucose quantity. The second sub-module 310 of the software module 306 further comprises a artificial neural network and error correction module 310g for combining the blood volume data and the blood glucose quantity to compute the concentration of the glucose in the blood.

In an embodiment, for the glucometer of the second embodiment, the data matrix is, for example, a three dimensional matrix with a dimension of [p][i][j], j=1, 2, . . . , 4 and corresponds to the number of detectors 110g-110j. The value of p=1, 2, or 3, and corresponds to the number of distinct wavelengths of the light emitting diodes. For example, p=1 when the first light emitting diode 108a with a wavelength of 650 nanometers is activated, p=2 when the second light emitting diode 108b with a wavelength of 940 nanometers is activated, and p=3 when the third light emitting diode 108a with a wavelength of 1160 nanometers is activated. In the above data matrix dimension, i=0, 1, 2, . . . , 1200. The detectors 110g-110j stop detecting the light for a selected light emitting diode (108a, 108b, or 108c) when the value of j=5.

In another embodiment, for the glucometer of the second embodiment, the data matrix is, for example, a three dimensional matrix with a dimension of [p][i][j]. The value of j=1, 2, . . . , 10 as described in the description of FIGS. 5A and 5B. However, the readings of cells [p][i][1], [p][i][2], [p][i][3], [p][i][4], [p][i][5], and [p][i][6] are set to zero in the data matrix. Only cells [p][i][7], [p][i][8], [p][i][9], and [p][i][10] that correspond to detectors 110g, 110h, 110i, and 110j are updated. The value of p=1, 2, or 3, and corresponds to the number of distinct wavelengths of the light emitting diodes. For example, p=1 when the first light emitting diode 108a with a wavelength of 650 nanometers is activated, p=2 when the second light emitting diode 108b with a wavelength of 940 nanometers is activated, and p=3 when the third light emitting diode 108a with a wavelength of 1160 nanometers is activated. In the above data matrix dimension, i=0, 1, 2, . . . , 1200. The detectors 110g-110j stop detecting the light for a selected light emitting diode (108a, 108b, or 108c) when the value of j=11.

In an embodiment, the method of non-invasively measuring concentration of glucose in blood further comprises detecting a photo plethysmograph from the finger tip for determining blood pressure along with the measurement of the concentration of the glucose in the blood. The glucometer 100 is also able to detect the photo plethysmograph from the finger-tip of the user alongside detecting the glucose readings while the detector and LED placement along with mechanical aspects of the glucometer 100 remain similar. Photo plethysmograph (PPG) is obtained using the NIR wavelength region (940 nm wavelength) to determine blood flow and blood pressure of the subject. The plethysmography wave is analysed for its Systolic Upstroke time (SUT), Diastolic time (DT) and Time delay between Systolic and diastolic peak (T₁). Systolic blood pressure (SBP) and Diastolic blood pressure (DBP) are determined using the above parameters of Photo plethysmography wave by using an algorithm within the software module 310e. The algorithm within the software module 310e is also able to determine blood flow from the plethysmography wave along with Systolic blood pressure and diastolic blood pressure. Blood pressure determination will help in categorizing the subject as hypotension or hypertension based on their determined SBP and DBP to give an alarm of his blood pressure condition along with blood glucose. The compensation algorithm when the Blood pressure changes will be used to make the device more accurate. Also advice the users to contact the doctor immediately when persistent signals of Hypo tension coupled with Hypo glycemia condition for example if the Hypotension <100/60 mm/Hg and blood glucose drops to 70 mg/dl, the device will advise the user to contact a doctor immediately.

The method of non-invasively measuring concentration of glucose in blood further comprises determining onset of diabetes in a non-diabetic user. To determine the onset of diabetes in the non-diabetic user, after the artificial neural network and error correction module 310g applies compensation coefficient algorithms and error detection, the computed concentration of the glucose in the blood is stored in a memory unit 304 of the glucometer 100. In case of error detected during the measurement, the glucometer 100 gives a warning to the user to repeat the test. The stored data is transmitted to a mobile device 324 through a Bluetooth module (not shown) in the glucometer 100. The mobile device 324 transmits the data to a cloud server 322 through a cellular network 320b. In an embodiment, the glucometer 100 is directly connected to a data network 320a though a wired or wireless network interface (not shown). The stored data is transmitted to the cloud server 322 through the data network 320a. The mobile device 324 and the cloud server 322 compile historical data for the user or for one or more users. The historical data is analyzed by the artificial intelligence running in the cloud server to provide diabetes prediction for the non-diabetic user. Thus, onset of diabetes in the non-diabetic user is determined. In an embodiment, the glucometer 100 and/or the mobile device 324 is configured to identify each of the one or more users and store the data in the cloud server 322 in a specific file created for each of the one or more users.

The glucometer 100 disclosed herein is designed to be user friendly and provide accurate glucose reading irrespective of hand movement and any other motion related errors. To achieve, in an embodiment, the glucometer 100 comprises an Accelerometer (not shown) that tracks the motion of the glucometer and predicts the motion artefact related noise and corrects the motion artefact related noise using Kalman filtering. Kalman filtering is a statistical method to correct measurement error arising due to random error sources. Kalman filtering technique is used to reduce noise from systems that are otherwise unpredictable.

The major sources of error are due to high absorbing species in blood, fatty tissue, skin and bone. The error arising due to absorbance by bone, skin and fatty tissue is minimized by using a technique called “photoplethysmography” where the pulsating part of the output signal of detector is present that is caused only due to the cardiac cycle and blood volume change that is periodic. The presence of different analytes in the blood also pose a measurement error, this is dealt with in the device because of the use of Near-Infrared spectroscopy concept. Since the NIR region of electromagnetic spectrum is minimally absorbed by water (in blood), melanin (skin pigment) and has a good amount of absorbance for glucose. The wavelength with such behaviour is 940 nm and 1160 nm.

Due to use of LEDs of multiple wavelengths more specific relationship between absorbance and glucose concentration can be found. The weighted sum of absorbance of both the wavelengths are being used to find the concentration of glucose:

C_{g} α (w₁*A₁+w₂*A₂), where w₁ and w₂ are weights and A₁ and A₂ are the absorbance at 940 nm and 1160 nm respectively, and ‘C_{g}’ is the concentration of glucose. The weights are calculated in the software module 310f.

Since haemoglobin is one of the major constituents of blood, it also contributes to the measurement error while measuring glucose. The wavelength 650 nm LED is used to compensate for the error due to haemoglobin content in blood. For isolating the haemoglobin error, haemoglobin also needs to be measured. Therefore, weighted average is also calculated for haemoglobin also.

Cₕα (w₃*A₁+w₄*A₃), where w₃ and w₄ are weights and A₁ and A₃ are the absorbance at 940 nm and 650 nm respectively, and ‘C_{g}’ is the concentration of glucose. The weights are calculated in the software module 310e.

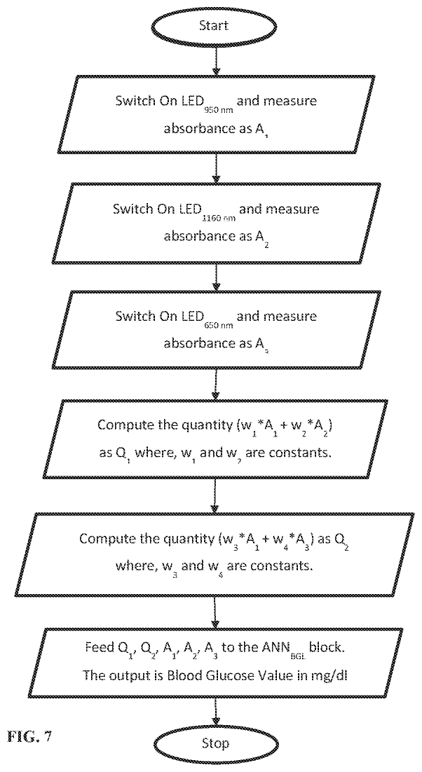

FIG. 7 illustrates a flow chart for applying the statistical processing of the absorbance data and displaying the concentration of blood glucose value as output. The glucometer 100 comprises an algorithm that automatically applies the statistical processing of the absorbance data and displays the concentration of blood glucose value as output. The flowchart of the algorithm is illustrated in FIG. 7.

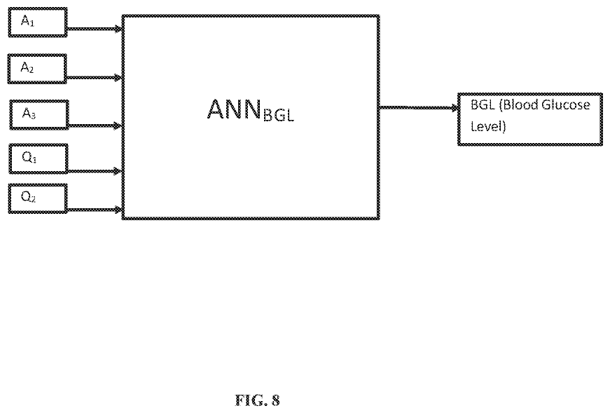

FIG. 8 illustrates the artificial neural network and error correction module 310g. The values of Q1, Q2, A1, A2, A3 illustrated in FIG. 7 are fed to the artificial neural network and error correction module 310g. The artificial neural network and error correction module 310g output the Blood Glucose Value in mg/dl.

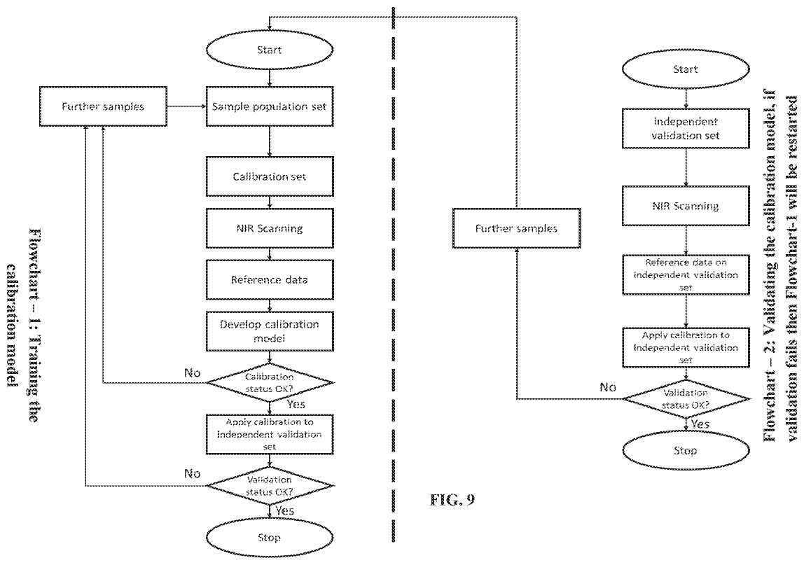

FIG. 9 illustrates an algorithm used to train the artificial neural network and error correction module 310g. Flowchart-1 illustrates the method of training the calibration model of the artificial neural network and error correction module 310g. Flowchart-2 illustrates the method of validating the calibration model. If validation fails then Flowchart-1 is restarted.

The foregoing examples have been provided merely for explanation and are in no way to be construed as limiting of the method and the glucometer 100 disclosed herein. While the method and the glucometer 100 have been described with reference to various embodiments, it is understood that the words, which have been used herein, are words of description and illustration, rather than words of limitation. Furthermore, although the method and the glucometer 100 have been described herein with reference to particular means, materials, and embodiments, the method and the glucometer 100 are not intended to be limited to the particulars disclosed herein; rather, the method and the glucometer 100 extend to all functionally equivalent structures, methods and uses, such as are within the scope of the appended claims. While multiple embodiments are disclosed, it will be understood by those skilled in the art, having the benefit of the teachings of this specification, that the method and the glucometer 100 disclosed herein are capable of modifications and other embodiments may be effected and changes may be made thereto, without departing from the scope and spirit of the method and the glucometer 100 disclosed herein.

We claim:

1. A glucometer (100), comprising:
a curved test surface (102) with a transparent test site (104) configured to accommodate a finger-tip when the finger-tip is placed on the transparent test site (104) for non-invasively measuring concentration of glucose in blood;
an enclosing lid (106) configured to enclose the finger-tip placed on the transparent test site;
a light source assembly (108) comprising a plurality of light emitting diodes (108a-108c) placed underneath the transparent test site (104);
a plurality of detectors (110a-110j) for detecting transflective light, transmissive light, and reflective light from blood capillaries at a depth of 100 micro meters below skin surface; and
a software module (306) comprising two sub-modules, wherein a first of the sub-modules (308) comprises a first set of algorithms for generating a data matrix, wherein each of the plurality of light emitting diodes is configured to be individually activated in a loop comprising 1200 cycles and the transflective light, the transmissive light, and the reflective light from the blood capillaries are measured and recorded in the data matrix of dimension (n×1200×m), wherein ‘n’ is a number of distinct wavelengths of light emitting diodes within the light source assembly and ‘m’ is a number of detectors, and wherein a second of the sub-modules (310) comprises a second set of algorithms for the non-invasive measurement of the concentration of the glucose in the blood by processing data within the data matrix.

2. The glucometer of claim 1, wherein initial value of all entries in the data matrix are set to zero.

3. The glucometer of claim 1, wherein the curved test surface (102) is hemispherical in shape.

4. The glucometer of claim 1, wherein the enclosing lid (106) is configured to apply pressure on the finger-tip placed on the transparent test site (104).

5. The glucometer of claim 1, wherein the ‘n’ is three, wherein a first of the distinct light emitting diodes is a red light emitting diode operating with a wavelength of 650 nanometers, wherein a second of the distinct light emitting diodes is a near-infrared light emitting diode operating at a wavelength of 940 nanometers, and wherein a third of the distinct light emitting diodes is a near-infrared light emitting diode operating at a wavelength of 1160 nanometers, wherein ‘m’ is ten, wherein the ten detectors comprise a first detector set comprising six detectors (110a-110f) for detecting the transflective light, a second detector set comprising a detector (110g) for detecting the transmissive light, and a third detector set comprising three detectors (110h-110j) for detecting the reflective light, wherein the detectors (110a-110f) in the first detector set are arranged along the curved test surface (102) on both sides of the transparent test site (104), wherein a seventh detector (110g) in the second detector set is accommodated in the enclosing lid (106) along a longitudinal axis perpendicular to a center of the transparent test site (104), and wherein the detectors (110h-110j) in the third detector set comprising an eighth detector (110h), a ninth detector (110i) and a tenth detector (110j) are accommodated within the light source assembly (108).

6. The glucometer of claim 5, wherein a first detector (110a) and a sixth detector (110f) of the first detector set are placed at an angle of −67.5° and +67.5° from the longitudinal axis linking the center of the transparent test site (104) and the seventh detector (110g), wherein a second detector (110b) and a fifth detector (110e) of the first detector set are placed at an angle of −45° and +45° from the longitudinal axis linking the center of the transparent test site (104) and the seventh detector (110g), and wherein a third detector (110c) and a fourth detector (110d) of the first detector set are placed at an angle of −22.5° and +22.5° from the longitudinal axis linking the center of the transparent test site (104) and the seventh detector (110g), wherein the eighth detector (110h) is co-located with the first light emitting diode 108a, wherein the ninth detector (110i) is co-located with the second light emitting diode 108b, wherein the tenth detector (110j) is co-located with the third light emitting diode 108c, wherein the seventh detector (110g) detects the transmissive light passing through the finger-tip and through blood capillaries at the depth of 100 micro meters below the skin surface, wherein the first detector (110a), the second detector (110b), the third detector (110c), the fourth detector (110d), the fifth detector (110e) and the sixth detector (110f) detect the transflective light reflected from the blood capillaries, and wherein the eighth detector (110h), the ninth detector (110i) and the tenth detector (110j) detect the reflective light from the blood capillaries.

7. The glucometer of claim 6, wherein the second sub-module (310) of the software module (306) comprises:
a third sub-module (310a) for combining data within the data matrix measured by the seventh detector (110g) at the wavelengths of 650 nanometers, 940 nanometers and 1160 nanometers to determine skin thickness correction data caused by variation in skin thickness;
a fourth sub-module (310b) for combining data within the data matrix measured by the first detector (110a), the second detector (110b), the third detector (110c), the fourth detector (110d), the fifth detector (110e), the sixth detector (110f) and the eighth detector (110h) at the wavelength of 650 nanometers;
a fifth sub-module (310c) for combining data within the data matrix measured by the first detector (110a), the second detector (110b), the third detector (110c), the fourth detector (110d), the fifth detector (110e), the sixth detector (110f) and the ninth detector (110i) at the wavelength of 940 nanometers;
a sixth sub-module (310d) for combining data within the data matrix measured by the first detector (110a), the second detector (110b), the third detector (110c), the fourth detector (110d), the fifth detector (110e), the sixth detector (110f) and the tenth detector (110j) at the wavelength of 1160 nanometers;
a seventh sub-module (310e) combining outputs of the third sub-module, the fourth sub-module, and the fifth sub-module 310c for processing one of blood volume data and blood pressure data; and
an eighth sub-module (310f) for combining outputs of the fourth sub-module 310b, the fifth sub-module 310c, and the sixth sub-module 310d to process blood glucose quantity.

8. The glucometer of claim 7, wherein the second sub-module (310) of the software module (306) further comprises an artificial neural network and error correction module (310g) for combining the blood volume data and the blood glucose quantity to compute the concentration of the glucose in the blood.

9. The glucometer of claim 8, wherein the computed concentration of the glucose in the blood is displayed on a display screen (318) of the glucometer (100).

10. The glucometer of claim 1, further comprising a guard member (122) configured to surround a right side (116a) and a left side (116b) of the body (116) of the glucometer (100) adjacent to the curved test surface (102) of the glucometer (100) to block the light from reaching the curved test surface (102) from the right side (116a) and the left side (116b) of the glucometer (100).

11. The glucometer of claim 10, wherein the guard member (122) is configured to slidably engage with the body (116) of the glucometer (100).

12.-14. (canceled)

15. A method of non-invasively measuring concentration of glucose in blood, comprising:
providing (402) a glucometer comprising a curved test surface with a transparent test site, an enclosing lid, a light source assembly, and a plurality of detectors for detecting transflective light, transmissive light, and reflective light from blood capillaries at a depth of 100 micro meters below skin surface, and a software module, wherein the light source assembly comprises a plurality of light emitting diodes placed underneath the transparent test site, wherein the software module comprises a first sub-module and a second sub-module, and wherein the first sub-module of the software module comprises a first set of algorithms for generating a data matrix, and wherein the second sub-module of the software module comprises a second set of algorithms for the non-invasive measurement of the concentration of the glucose in the blood by processing data within the data matrix;
applying pressure (404) to a finger-tip placed on the transparent test site by closing the enclosing lid on the finger-tip;
activating (406) each of the plurality of light emitting diodes individually in a loop comprising 1200 cycles;
measuring (408) the transflective light, the transmissive light and the reflective light from the blood capillaries within the pressurized finger-tip, and recording the measurements in the data matrix of dimension (n×1200×m), where ‘n’ is a number of distinct wavelengths of the light emitting diodes within the light source assembly and ‘m’ is a number of detectors; and
measuring (410) the concentration of the glucose in the blood by the processing of the data within the data matrix.

16. (canceled)

17. The method of claim 15, wherein the ‘n’ is three, wherein a first of the distinct light emitting diodes is a red light emitting diode operating with a wavelength of 650 nanometers, wherein a second of the distinct light emitting diodes is a near-infrared light emitting diode operating at a wavelength of 940 nanometers, and wherein a third of the distinct light emitting diodes is a near-infrared light emitting diode operating at a wavelength of 1160 nanometers, wherein the ‘m’ is ten, wherein the ten detectors comprise a first detector set comprising six detectors (110a-110f) for detecting the transflective light, a second detector set comprising a detector (110g) for detecting the transmissive light, and a third detector set comprising three detectors (110h-110j) for detecting the reflective light, wherein the detectors (110a-110f) in the first detector set are arranged along the curved test surface (102) on both sides of the transparent test site (104), wherein a seventh detector (110g) in the second detector set is accommodated in the enclosing lid (106) along a longitudinal axis perpendicular to a center of the transparent test site (104), and wherein the detectors (110h-110j) in the third detector set comprising an eighth detector (110h), a ninth detector (110i) and a tenth detector (110j) are accommodated within the light source assembly (108).

18. The method of claim 17, wherein a first detector (110a) and a sixth detector (110f) of the first detector set are placed at an angle of −67.5° and +67.5° from the longitudinal axis linking the center of the transparent test site (104) and the seventh detector (110g), wherein a second detector (110b) and a fifth detector (110e) of the first detector set are placed at an angle of −45° and +45° from the longitudinal axis linking the center of the transparent test site (104) and the seventh detector (110g), and wherein a third detector (110c) and a fourth detector (110d) of the first detector set are placed at an angle of −22.5° and +22.5° from the longitudinal axis linking the center of the transparent test site (104) and the seventh detector (110g), wherein the eighth detector (110h) is co-located with the first light emitting diode 108a, wherein the ninth detector (110i) is co-located with the second light emitting diode 108b, wherein the tenth detector (110j) is co-located with the third light emitting diode 108c, wherein the seventh detector (110g) detects the transmissive light passing through the finger-tip and through blood capillaries at the depth of 100 micro meters below the skin surface, wherein the first detector (110a), the second detector (110b), the third detector (110c), the fourth detector (110d), the fifth detector (110e) and the sixth detector (110f) detect the transflective light reflected from the blood capillaries, and wherein the eighth detector (110h), the ninth detector (110i) and the tenth detector (110j) detect the reflective light from the blood capillaries.

19. The method of claim 18, further comprising:
combining data within the data matrix measured by the seventh detector (110g) at the wavelengths of 650 nanometers, 940 nanometers and 1160 nanometers to determine skin thickness correction data caused by variation in skin thickness, by a third sub-module (310a) of the second sub-module (310) of the software module (306);
combining data within the data matrix measured by the first detector (110a), the second detector (110b), the third detector (110c), the fourth detector (110d), the fifth detector (110e), the sixth detector (110f) and the eighth detector (110h) at the wavelength of 650 nanometers, by a fourth sub-module (310b) of the second sub-module (310) of the software module (306);
combining data within the data matrix measured by the first detector (110a), the second detector (110b), the third detector (110c), the fourth detector (110d), the fifth detector (110e), the sixth detector (110f) and the ninth detector (110i) at the wavelength of 940 nanometers, by a fifth sub-module (310c) of the second sub-module (310) of the software module (306);
combining data within the data matrix measured by the first detector (110a), the second detector (110b), the third detector (110c), the fourth detector (110d), the fifth detector (110e), the sixth detector (110f) and the tenth detector (110j) at the wavelength of 1160 nanometers, by a sixth sub-module (310d) of the second sub-module (310) of the software module (306);
combining outputs of the third sub-module (310a), the fourth sub-module (310b), and the fifth sub-module (310c) to process one of blood volume data and blood pressure data, by a seventh sub-module (310e) of the second sub-module (310) of the software module (306); and
combining outputs of the fourth sub-module (310b), the fifth sub-module (310c), and the sixth sub-module (310d) to process blood glucose quantity, by an eighth sub-module (310f) of the second sub-module (310) of the software module (306).

20. (canceled)

21. The method of claim 20, further comprising:
applying compensation coefficient algorithms (414) to mitigate effects of temperature, humidity, and motion artifacts, by said artificial neural network and error correction module (310g); and
applying error detection (416), by said artificial neural network and error correction module (310g), before the computed concentration of the glucose in the blood is displayed on the display screen (310h) of the glucometer (100).

22. The method of claim 21, further comprising:
storing (416) the computed concentration of the glucose in the blood in a memory of the glucometer (100); and
transmitting (418) the stored data to a mobile device and to a cloud server for further prognosis.

23. A glucometer (100), comprising:
a curved test surface (102) with a transparent test site (104) configured to accommodate a finger-tip when the finger-tip is placed on the transparent test site (104) for non-invasively measuring concentration of glucose in blood;
an enclosing lid (106) configured to enclose the finger-tip placed on the transparent test site (104);
a light source assembly (108) comprising a plurality of light emitting diodes (108a-108c) placed underneath the transparent test site (104);
a plurality of detectors (110g-110j) for detecting transmissive light (110g) and reflective light (110h-110j) from blood capillaries at a depth of 100 micro meters below skin surface; and
a software module (306) comprising two sub-modules, wherein a first of the sub-modules (308) comprises a first set of algorithms for generating a data matrix, wherein each of the plurality of light emitting diodes is configured to be individually activated in a loop comprising 1200 cycles and the transmissive light and the reflective light from the blood capillaries are measured and recorded in the data matrix of dimension (n×1200×m), wherein ‘n’ is a number of light emitting diodes within the light source assembly, wherein ‘m’ is a number of detectors, and wherein a second of the sub-modules (310) comprises a second set of algorithms for the non-invasive measurement of the concentration of the glucose in the blood by processing data within the data matrix.

24.-29. (canceled)

30. The method of claim 20, further comprising detecting a photo plethysmograph from the finger tip for determining blood pressure along with the measurement of the concentration of the glucose in the blood.

31. The method of claim 30, wherein the photo plethysmograph is obtained using the near-infrared light emitting diode operating at a wavelength of 940 nanometers to determine the blood pressure, and wherein a plethysmography wave is analyzed for its Systolic Upstroke time (SUT), Diastolic time (DT), and Time delay between Systolic and diastolic peak (T₁).

32. (canceled)
